(19) United States
(12) Patent Application Publication
(10) Pub. No.: US 2016/0312175 A1
(43) Pub. Date: Oct. 27, 2016

(54) Cell coating method using compound comprising gallate group and lanthanoid metal salt or transition metal salt

(71) Applicant: Korea Advanced Institute of Science and Technology, Daejeon (KR)

(72) Inventors: Insung Choi, Daejeon (KR); Ji Hun Park, Daejeon (KR); Younghoon Lee, Daejeon (KR); Kyunghwan Kim, Seoul (KR); Juno Lee, Chuncheon-si (KR)

(73) Assignee: Korea Advanced Institute of Science and Technology, Daejeon (KR)

(21) Appl. No.: 14/854,583

(22) Filed: Sep. 15, 2015

(30) Foreign Application Priority Data
Apr. 21, 2015 (KR) 10-2015-0056109

(51) Int. Cl. C12N 1/04; C12N 5/00

(52) U.S. Cl. CPC C12N 1/04; C12N 5/0006

(57) ABSTRACT

The present invention relates to a cell coating method using a compound including a gallate group, and a lanthanoid metal salt or transition metal salt. According to the cell coating method of the present invention, a cell having a surface coated with a nanoshell prepared by this method is stably protected from external environmental stimulus such as light irradiation, and silver nanoparticles, and the coating is degraded as necessary without damaging the cell.

### CROSS-REFERENCES TO RELATED APPLICATION

This patent application claims the benefit of priority from Korean Patent Application No. 10-2015-0056109, filed on Apr. 21, 2015, the contents of which are incorporated herein by reference.

### BACKGROUND OF THE INVENTION

1. Field of the Invention

The present disclosure relates to a cell coating method using a compound including a gallate group, and a lanthanoid metal salt or transition metal salt.

2. Description of the Related Art

Some bacteria such as bacillus, chlostridia, and sporosarcina respond to external environment, and thus sporulation and germination, which are mutually organized biological processes, occur.

Sporulation is cell differentiation which blocks cell metabolism and forms a proteinaceous shell to counteract external stress factors such as malnutrition, dehydration, heat and radiation. The shell takes a role in protecting an inside from invasion of external materials and detoxifying active toxic chemicals. The shell for protecting cells is degraded when a spore's inner membrane senses environment suitable for propagation, and this process is referred to as germination.

Since most of living cells are extremely weak in laboratories, substantial application is difficult. To solve the limitation, a study has been conducted to increase in vitro stability of cells by structurally mimicking the sporulation process due to cell level adaptation to naturally occurring environment (i.e. to chemically form an ultrathin artificial shell on the non-spore forming cells).

In the typical cell-coating study field, non-patent document 1 (S. H. Yang, D. Hong, J. Lee, E. H. Ko, I. S. Choi, Small 2013, 9, 178-186) discloses that mimicking of the sporulation process allows an artificial shell having an excellent durability to enhance cell resistance to external stress factors. In addition, it has been known that a nanoshell of silica, silica-titania, graphene or polydopamine may be formed on microorganism and mammalian cells, thereby enhancing resistance to physiochemical stress factors, malnutrition, enzyme attack or heat.

However, non-patent document 2 (T. M. S. Chang, Nat. Rev. Drug Discovery 2005, 4, 221-235) discloses a problem of development of the chemical method mimicking the germination process, that is, due to the degradable property of the shell, although programming is performed focused on the degradation of the shell, it is difficult to apply the cells to sensors, drug delivery system, cell therapy, or regenerative medicine.

When the shell does not respond to changes in external environment, the shell often acts as a physical barrier against an intracellular biological action of the coated cells. Thus, forming a shell, which has excellent degradability as necessary, is an essential factor for efficient application for a cell loaded material and device areas. However, typical strategies to degrade a material having physicochemically excellent durability require toxic chemicals and deteriorated conditions which lead cell death.

Therefore, during conducting a study based on the fact that an organometallic shell based on a non covalent coordination complex is structurally stable and as well as degradable under environment suitable for cell survival by responding to external stimulus, the present inventors have completed the present invention by demonstrating that, according to the cell coating method of the present invention, the cell having a surface coated with a nanoshell prepared by this method is stably protected from external environmental stimulus such as light irradiation, and silver nanoparticles, and the coating is degraded as necessary without damaging cells.

### PRIOR ART DOCUMENT

### Non-Patent Document

(Non-patent document 1) S. H. Yang, D. Hong, J. Lee, E. H. Ko, I. S. Choi, Small 2013, 9, 178-186.

(Non-patent document 2) T. M. S. Chang, Nat. Rev. Drug Discovery 2005, 4, 221-235.

### SUMMARY OF THE INVENTION

One object of the present invention is to provide a cell coating method including (step 1) adding, to an aqueous solution including cells, a compound having one or more gallate groups expressed by Chemical Formula

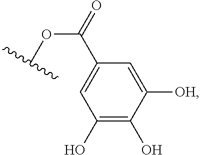 [SMILES: *OC(=O)c1cc(O)c(O)c(O)c1]

and a lanthanoid metal salt or transition metal salt.

Another object of the present invention is to provide a cell having a surface coated with a nanoshell including a compound having one or more gallate groups expressed by Chemical Formula

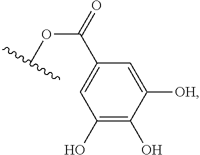 [SMILES: *OC(=O)c1cc(O)c(O)c(O)c1]

and lanthanoid metal ion or transition metal ion.

Still another object of the present invention is to provide a cell protecting method including (step 1) adding, to an aqueous solution including cells, a compound having one or more gallate groups expressed by Chemical Formula

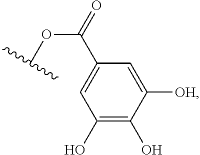 [SMILES: *OC(=O)c1cc(O)c(O)c(O)c1]

and a lanthanoid metal salt or transition metal salt.

Even another object of the present invention is to provide a cell proliferation inhibiting method including (step 1) adding, to an aqueous solution including cells, a compound having one or more gallate groups expressed by Chemical Formula

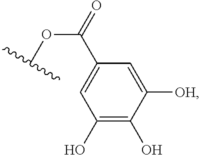 [SMILES: *OC(=O)c1cc(O)c(O)c(O)c1]

and a lanthanoid metal salt or transition metal salt.

In order to achieve the objects, the present invention provides a cell coating method including (step 1) adding, to an aqueous solution including cells, a compound having one or more gallate groups expressed by Chemical Formula

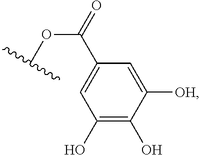 [SMILES: *OC(=O)c1cc(O)c(O)c(O)c1]

and a lanthanoid metal salt or transition metal salt.

The present invention also provides a cell having a surface coated with a nanoshell including a compound having one or more gallate groups expressed by Chemical Formula

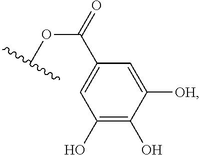 [SMILES: *OC(=O)c1cc(O)c(O)c(O)c1]

and lanthanoid metal ion or transition metal ion.

Furthermore, the present invention provides a cell protecting method including (step 1) adding, to an aqueous solution including cells, a compound having one or more gallate groups expressed by Chemical Formula

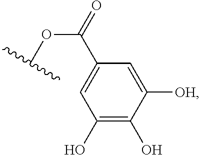 [SMILES: *OC(=O)c1cc(O)c(O)c(O)c1]

and a lanthanoid metal salt or transition metal salt.

The present invention also provides a cell proliferation inhibiting method including (step 1) adding, to an aqueous solution including cells, a compound having one or more gallate groups expressed by Chemical Formula

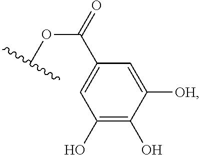 [SMILES: *OC(=O)c1cc(O)c(O)c(O)c1]

and a lanthanoid metal salt or transition metal salt.

According to the cell coating method of the present invention, cells having surfaces coated with nanoshells prepared by this method are stably protected from external environmental stimulus such as light irradiation and silver nanoparticles, and the coating is degraded as necessary without damaging cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an image schematically showing a process of coating Saccharomyces cerevisiae (TA-Fe^{III} shell), and then degrading the coated shell as described in following Example 1.

FIG. 2 is an image showing a result of evaluating cell viability through Synerge™ MX multi-mode microplate reader (BioTekInstruments, USA) to evaluate whether yeast cells coated with TA-Fe(III) shell prepared in Example 1 show an excellent viability after coating.

FIG. 3 is a raman spectrum image to evaluate whether the TA-Fe(III) shell prepared in Example 1 is well coated on yeast cells.

FIG. 4 is an image showing a structure observed through scanning electron microscopy (SEM) and transmission electron microscopy (TEM) to evaluate whether TA-Fe(III) shell prepared in Example 1 is well coated on yeast cells.

FIG. 5 is an image showing presence and absence of cell aggregation through LSM 700 confocal laser-scanning microscopy (Carl Zeiss, Germany) to evaluate whether yeast cells coated with TA-Fe(III) shell prepared in Example 1 are aggregated after coating due to E. coli.

FIG. 6 is an image observed through confocal laser-scanning microscopy (Carl Zeiss, Germany) after inducing binding of cells to BSA-Alexa 647 (0.4 mg·mL⁻¹, Life Technologies), which is a protein conjugated with a chromophore, to evaluate a protein conjugation ability of TA-Fe(III) shell prepared in Example 1.

FIG. 7 is an image observed through confocal laser-scanning microscopy (Carl Zeiss, Germany) after inducing binding of cells to BSA-Alexa 647 (0.4 mg·mL⁻¹, Life Technologies), which is a protein conjugated with a chromophore, and adding fluorescein diacetate (FDA, Sigma) to evaluate a protein conjugation ability of the TA-Fe(III) shell prepared in Example 1, and also evaluate viability of the cells coated with the shell.

FIG. 8 is an image showing a cross section of [TA-Fe^{III}]₂, i.e., TA-Fe(III) shell prepared in Comparative Example 1 observed through TEM, indicating a thickness of about 20 nm.

FIG. 9 is an image showing a result of conducting an experiment to evaluate whether cell differentiation ability of yeast cells prepared in Example 1 is adjusted after coating, wherein the yeast cells are coated with [TA-Fe^{III}]₄, i.e., TA-Fe(III) shell.

FIG. 10 is an image showing a result of evaluating whether, in the yeast cells coated with [TA-Fe^{III}]₄, i.e., TA-Fe(III) shell, prepared in Example 1, cells are entirely protected from UV irradiation after coating.

FIG. 11 is an image showing a result of evaluating whether, in the yeast cells coated with [TA-Fe^{III}]₄, i.e., TA-Fe(III) shell, prepared in Example 1, cells are entirely protected from silver nanoparticles after coating.

FIG. 12 is an image showing a structure observed through SEM image to evaluate coating is good in HeLa cells coated with [TA-Fe^{III}]₄, i.e., TA-Fe(III) shell, prepared in Example 2.

FIG. 13 is an image showing viability of HeLa cells after coating evaluated through Live/Dead® viability/cytotoxicity kit (Life Technologies), wherein the HeLa cells coated with [TA-Fe^{III}]₄, i.e., TA-Fe(III) shell prepared in Example 2.

FIG. 14 is an image showing a red blood cell coated with stable TA-Fe(III) nanoshell prepared in Example 3.

FIG. 15 is an image showing a T lymphocyte coated with stable TA-Fe(III) nanoshell prepared in Example 4.

FIG. 16 is an image showing a fibroblast coated with stable TA-Fe(III) nanoshell prepared in Example 5.

### DESCRIPTION OF THE PREFERRED EMBODIMENTS

Hereinafter, the present invention will be described in more detail.

The present invention relates to a cell coating method including (step 1) adding, to an aqueous solution including cells, a compound having one or more gallate groups expressed by Chemical Formula

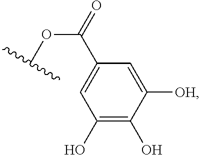 [SMILES: *OC(=O)c1cc(O)c(O)c(O)c1]

and lanthanoid metal salt or transition metal salt.

Hereinafter, the cell-coating method according to the present invention will be described in more detail in stepwise fashion.

In the cell coating method according to the present invention, step 1 is adding, to an aqueous solution including cells, a compound having one or more qallate groups expressed by Chemical Formula

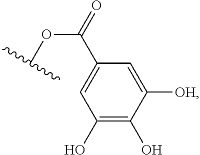 [SMILES: *OC(=O)c1cc(O)c(O)c(O)c1]

and a lanthanoid metal salt, or transition metal salt.

Available cells may include yeast cells, mammalian cells, or immune cells having a metabolic activity, and more particularly, Saccharomyces cerevisiae cells, HeLa cells, red blood cells, T lymphocytes, and NIH3T3 cells, etc. may be used.

In addition, as the aqueous solution, any solution capable of keeping cells alive may be used without particular limitation. More particularly, deionized water, phosphate buffer saline (PBS), Fetal bovine serum (FBS), Dulbecco's Modified Eagle's medium (DMEM), RPMI Media 1640, etc. may be used.

Further, as the compound having a gallate group, any compound having a gallate group may be used without particular limitation. More particularly, tannic acid (TA), gallic acid, theaflavin-3-gallate, epigallocatechin gallate, and epicatechin gallate, etc, may be used. Most preferably, TA may be used.

Additionally, as the metal of the lanthanoid metal salt, cerium (Ce), europium (Eu), gadolinium (Gd) and terbium (Tb), etc. may be used.

Further, as the metal of the transition metal salt, aluminum (Al), vanadium (V), manganese (Mn), ferrous (Fe), zinc (Zn), zirconium (Zr), molybdenum (Mo), ruthenium (Ru) and rhodium (Rh), etc. may be used.

In addition, Cl⁻, NO₃²⁻, CH₃CO₂⁻, and PO₄³⁻, etc. may be used as an anion capable of forming a salt with the metal of the lanthanoid metal salt or the metal of the transition metal salt without particular limitation.

The cell coating method may further include (step 2) purifying the coated cells through centrifugation after step 1. A thickness of the coating may be adjusted by repetitively performing the cell coating method which further includes step 2. The number of repetition is preferably 2 to 6, more preferably 3 to 5, and most preferably 4. In the case where the cell coating protocol is performed once, there is a problem in that a thickness of the shell coated on the cell surface is insufficient to protect the cells. In the case where the cell coating protocol is performed more than six times, there is a problem in that the thickness of the shell coated on the cell surface becomes thick more than needed, so that degradability of the coating is reduced.

In the cell coating method, before (step 2) purifying coated cell through centrifugation, pH buffer is additionally added to stabilize pH.

As the pH buffer, 3-(N-morpholino)propanesulfonic acid (MOPS) buffer may be used. The buffer is added until pH of the mixture prepared in step 1 preferably becomes 7.0-8.0, and most preferably becomes 7.4.

Further, the present invention provides a cell having a surface coated with a nanoshell including a compound having one or more gallate groups expressed by Chemical Formula

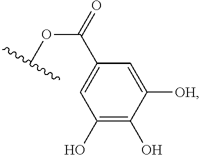 [SMILES: *OC(=O)c1cc(O)c(O)c(O)c1]

and a lanthanoid metal ion or transition metal ion.

Preferably, as the lanthanoid metal ion or transition metal ion, those capable of forming a complex with a gallate group expressed by Chemical Formula

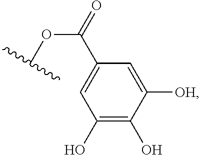 [SMILES: *OC(=O)c1cc(O)c(O)c(O)c1]

are used.

The nanoshell thickness is preferably 25 to 55 nm, more preferably 30 to 50 nm, and most preferably 40 nm. The nanoshell thickness of less than 25 nm is problematic in that the thickness of the shell coated on the cell surface is insufficient thickness to protect the cells. The nanoshell thickness of more than 55 nm is problematic in that the thickness of shell coated on the cell surface becomes thick more than needed, so that degradability of the coating is reduced.

Further, the present invention provides a cell protecting method including (step 1) adding, to an aqueous solution including cells, a compound having one or more gallate groups expressed by Chemical Formula

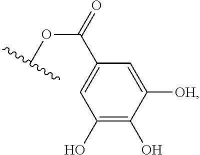 [SMILES: *OC(=O)c1cc(O)c(O)c(O)c1]

and a lanthanoid metal salt or transition metal salt.

The cell protecting method is characterized in that cells are kept alive and protected from external environment by a film coated on the cell surface, wherein the external environment means external environmental stimulus such as light irradiation, silver nanoparticles, and heat.

In addition, the present invention provides a cell proliferation inhibiting method including (step 1) adding, to an aqueous solution including cells, a compound having one or more gallate groups expressed by Chemical Formula

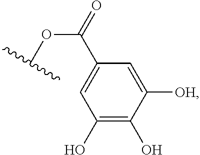 [SMILES: *OC(=O)c1cc(O)c(O)c(O)c1]

and a lanthanoid metal salt or transition metal salt.

The cell proliferation inhibiting method is characterized in that cells are kept alive and cell proliferation is inhibited by a film coated on the cell surface.

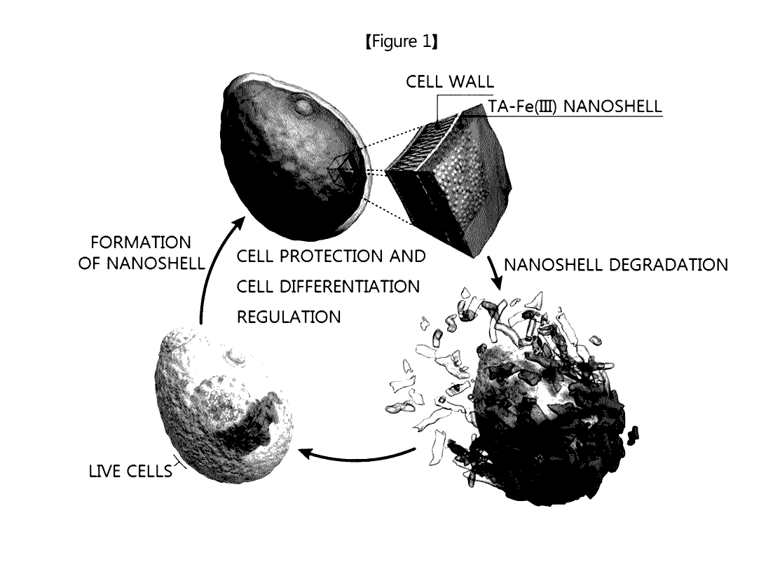

According to the cell coating method of the present invention, the cell having the surface coated with the nanoshell prepared by this method is stably protected from external environmental stimulus such as light irradiation and silver nanoparticles, and the coating is degraded as necessary without damaging cells. In particular, a substrate-independent coating (TA-Fe^{III} shell), which uses a coordination complex of TA and Fe^{III} ion, is highly biocompatible, and degradation proceeds rapidly within several seconds (see FIG. 1).

FIG. 1 is an image schematically showing a process of coating Saccharomyces cerevisiae (TA-Fe^{III} shell), and then degrading the coated shell as described in following Example 1.

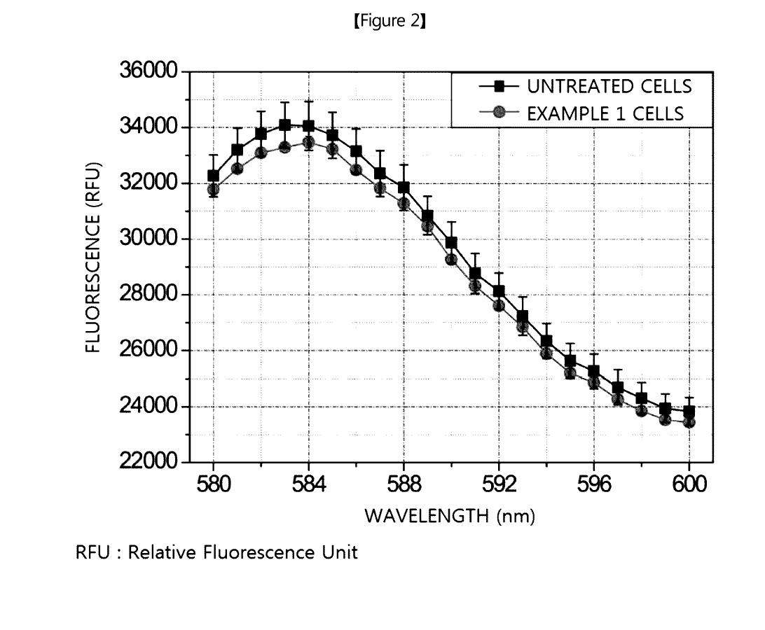

Thus, an experiment is conducted to evaluate whether yeast cells coated with [TA-Fe^{III}]₄, i.e. TA-Fe(III) shell, prepared in Example 1 show excellent viability after coating. Consequently, the yeast cells coated with TA-Fe(III) shell prepared in Example 1 have esterase similar to that of baker's yeast cells which are not treated at all, indicating that viability is maintained (see FIG. 2 of Experimental Example 1).

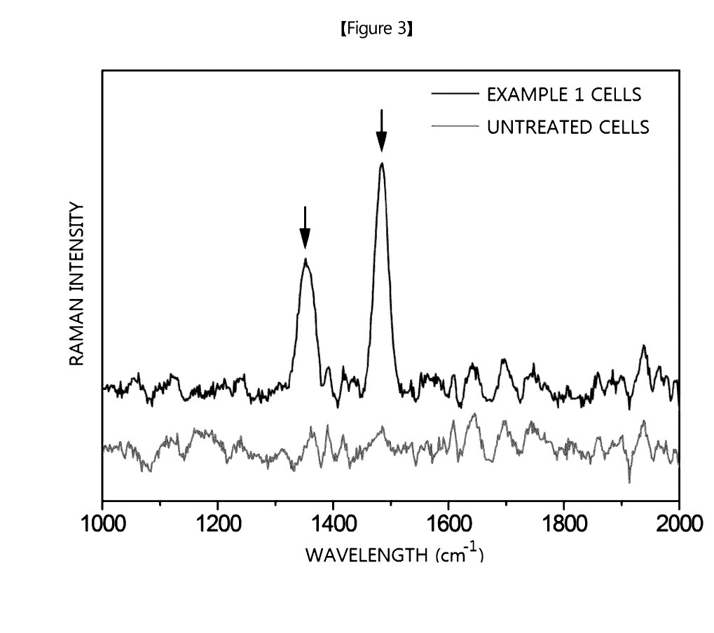

In addition, to evaluate whether [TA-Fe^{III}]₄, i.e. TA-Fe(III) shell, prepared in Example 1 is well coated on yeast cells, a raman spectrum is obtained by using Jobin Yvon/HORIBA LabRAM spectrometer equipped with a microscopy (Olympus BX 41, Japan). Consequently, it has been found that strong bands appear at 1354 cm⁻ and 1482 cm⁻ wavelength areas, indicating TA having a ring structure coated on the yeast cells, so that TA-Fe(III) shell prepared in Example 1 is well coated on the yeast cells (see FIG. 3 of Experimental Example 2).

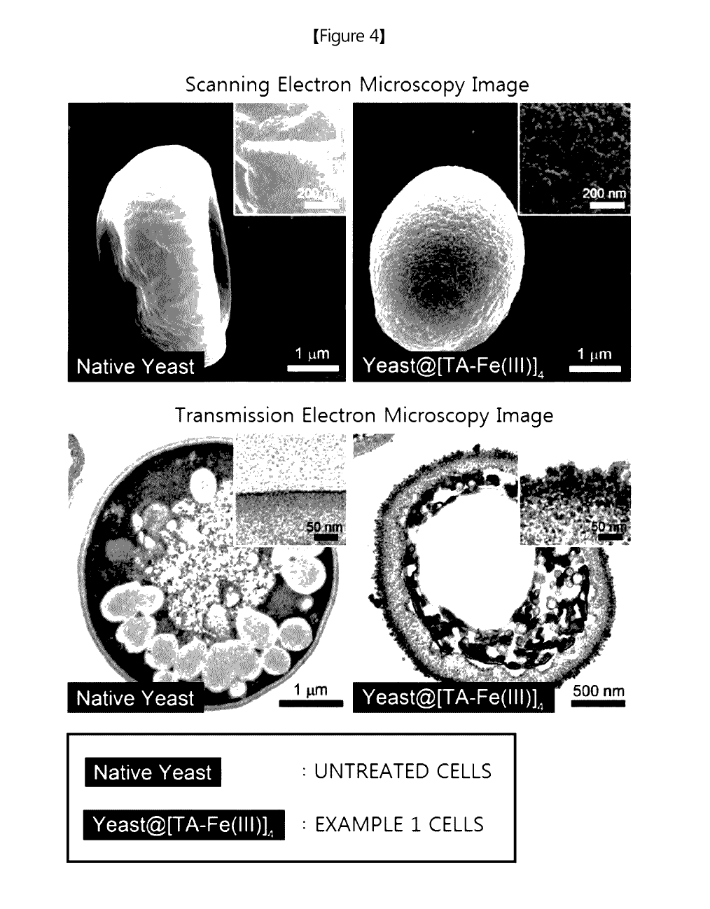

Further, to evaluate whether [TA-Fe^{III}]₄, i.e. TA-Fe(III) shell, prepared in Example 1 is well coated on yeast cells, a structure is observed through scanning electron microscopy (SEM) and transmission electron microscopy (TEM). Consequently, it has been shown that, as shown in the SEM image, comparing to untreated cells, homogeneous TA-Fe(III) shells are formed on the overall cells of Example 1. Additionally, as shown in the TEM image, by dissecting the cells to observe a cross section, it has been found that the average thickness of TA-Fe(III) shells is 40 nm (see FIG. 4 of Experimental Example 3).

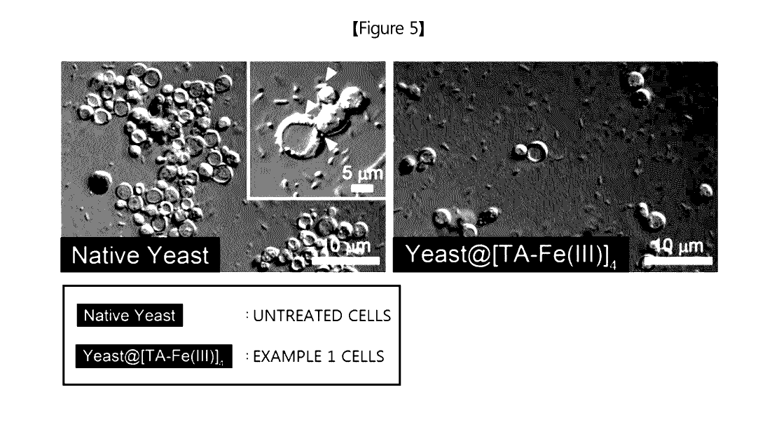

In addition, an experiment is conducted to evaluate whether, after coating, cell aggregation due to E. coli occurs in yeast cells coated with [TA-Fe^{III}]₄, i.e. TA-Fe(III) shell, prepared in Example 1. Consequently, it has been shown that, for untreated cells, cell aggregation due to E. coli occurs, whereas, for the cells prepared in Example 1, cell aggregation do not occur due to increased surface negative charges (see FIG. 5 of Experimental Example 4).

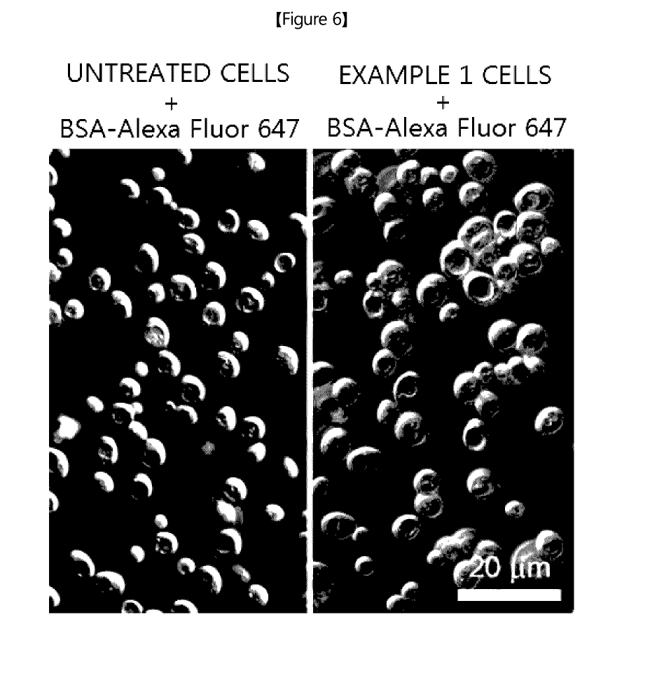

Further, an experiment is conducted to evaluate protein conjugation ability of [TA-Fe^{III}]₄, i.e. TA-Fe(III) shell, prepared in Example 1. Consequently, it has been found that, for untreated cells, there is no shell capable of binding to BSA-Alexa 647, which is a protein conjugated with a chromophore, so that fluorescence is not observed, whereas, for the cells prepared in Example 1, TA-Fe(III) shells bind to BSA-Alexa 647 so that fluorescence is observed, which indicates excellent protein conjugation ability (see FIG. 6 of Experimental Example 5).

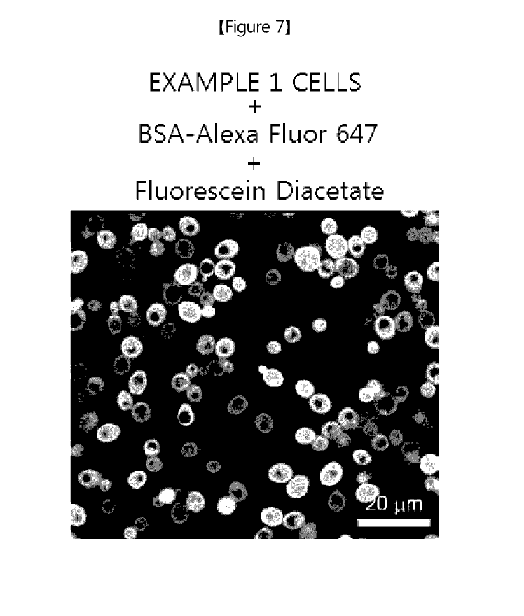

Moreover, in addition to protein conjugation ability of cells prepared in Example 1, to evaluate viability, fluorescein diacetate (FDA, Sigma) analysis is performed. Consequently, it has been found that, since a core-shell structure for live cells is shown, the cells prepared in Example 1 have excellent viability, as well as protein conjugation ability (see FIG. 7 of Experimental Example 5).

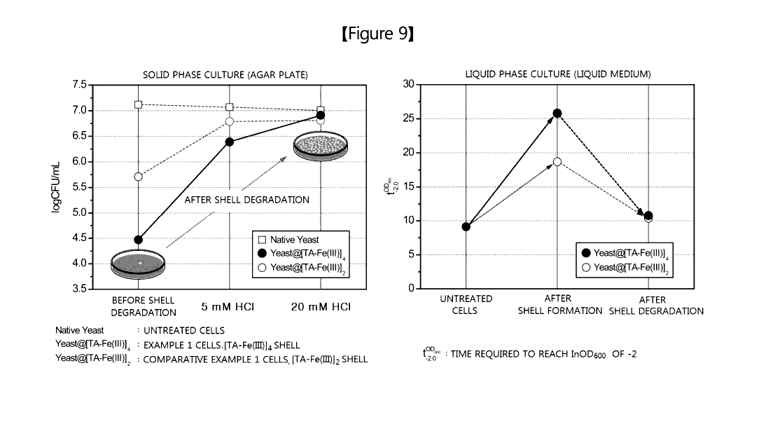

Further, an experiment is conducted to evaluate whether cell differentiation ability of yeast cells prepared in Example 1 is regulated after coating, wherein the yeast cells are coated with [TA-Fe^{III}]₄ i.e. TA-Fe(III) shell. Consequently, as shown in solid-phase culture (agar plate) of FIG. 9, it has been found that, before an acid is added, a colony-forming unit (CFU) value of the cells coated with the shell [TA-Fe^{III}]₄ (thickness of about 40 nm) prepared in Example 1 is significantly lower than those of untreated cells and Comparative Example 1 cells ([TA-Fe^{III}]₂ having a thickness of about 20 nm), which indicates that cell differentiation ability is readily inhibited. Additionally, it has been found that, when the shell prepared in Example 1 is degraded by adding an acid, cell differentiation same as in untreated cells occurs.

In addition, as shown in liquid-phase culture (liquid medium) of FIG. 9, it has been shown that time required to achieve InOD₆₀₀ of −2 increases in proportion to the thickness of the shell coated on cells. Namely, it has been found that cell differentiation of the cells coated with the shell [TA-Fe^{III}]₄ (thickness of about 40 nm) prepared in Example 1 is significantly inhibited comparing to untreated cells and Comparative Example 1 cells (see FIG. 9 of Experimental Example 6).

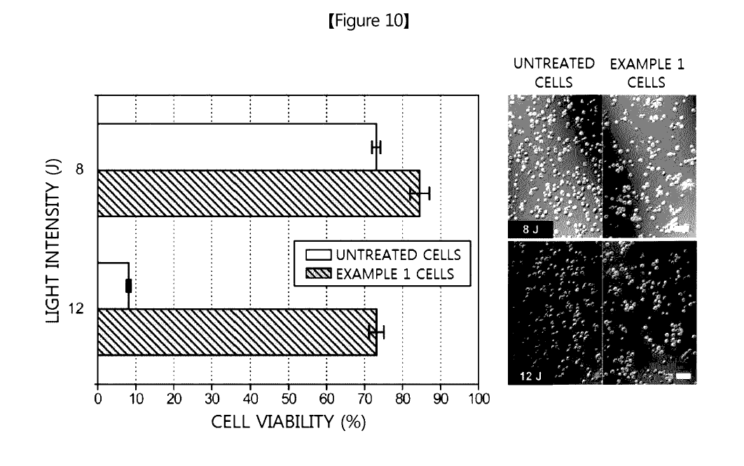

Further, an experiment is conducted to evaluate whether cells are entirely protected from UV irradiation after coating in the yeast cells coated with [TA-Fe^{III}]₄, i.e. TA-Fe(III) shell, prepared in Example 1. Consequently, comparing to untreated cells, it has been shown that cell viability from UV irradiation of the cells prepared in Example 1 is significantly increased due to [TA-Fe^{III}]₄ i.e. TA-Fe(III) shell. In particular, when light having the intensity of 12 J is irradiated, it has been shown that viability of untreated cells is about 9%, whereas viability of cells (i.e. cells prepared in Example 1) coated with [TA-Fe^{III}]₄, i.e. TA-Fe(III) shell, is 70% or more (see FIG. 10 of Experimental Example 7).

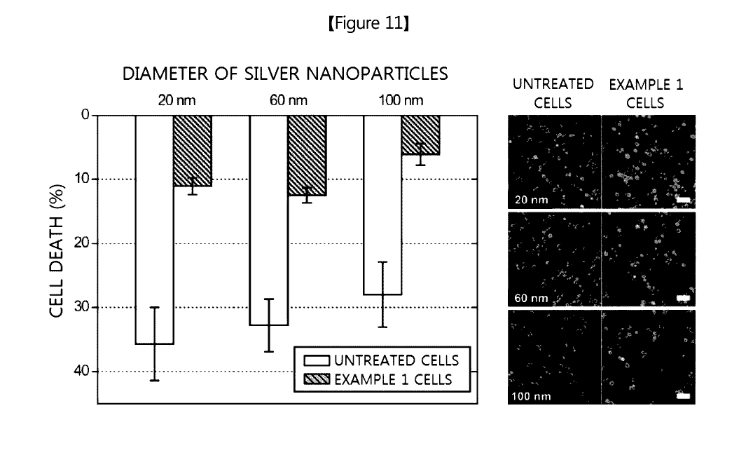

Additionally, an experiment is conducted to evaluate whether yeast cells are entirely protected from silver nanoparticles after coating in the yeast cells coated with [TA-Fe^{III}]₄, i.e. TA-Fe(III) shell, prepared in Example 1. Consequently, it has been shown that cell viability from silver nanoparticles of the cells prepared in Example 1 is significantly increased due to [TA-Fe^{III}]₄, i.e. TA-Fe(III) shell. In particular, it has been found that, when silver nanoparticles (diameter: 20 nm, 60 nm, or 100 nm) are added, untreated cells show cell death (%) of about 28% or more, whereas cells (i.e. cells prepared in Example 1) coated with [TA-Fe^{III}]₄, i.e. TA-Fe(III) shell, show cell death (%) of about 11% or less (see FIG. 11 of Experimental Example 8).

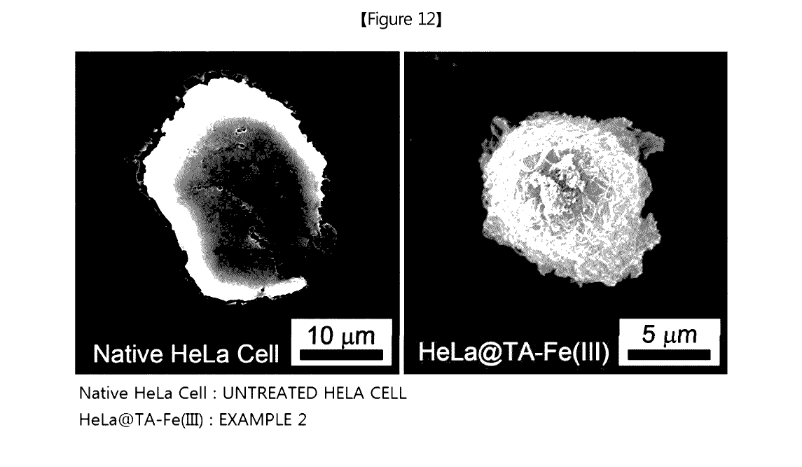

Further, an experiment is conducted to evaluate quality of coating in HeLa cells coated with [TA-Fe^{III}]₄, i.e. TA-Fe(III) shell, prepared in Example 2. Consequently, comparing to untreated HeLa cells, it has been found that homogeneous TA-Fe(III) shells are formed on the overall HeLa cells of Example 2 (see FIG. 12 of Experimental Example 9).

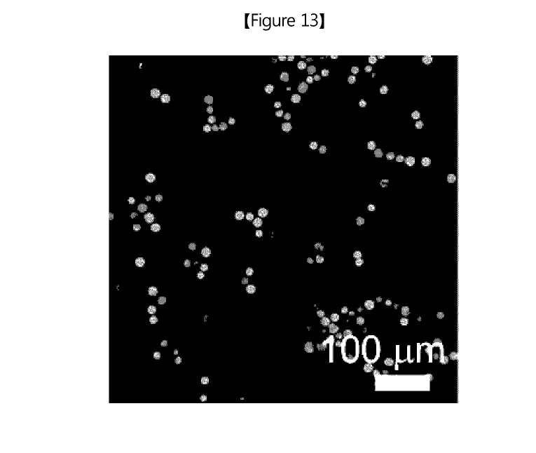

In addition, an experiment is conducted to evaluate whether the HeLa cells coated with [TA-Fe^{III}]₄, i.e. TA-Fe(III) shell, prepared in Example 2 show an excellent viability after coating. Consequently, it has been shown that, for the HeLa cells coated with [TA-Fe^{III}]₄, i.e. TA-Fe(III) shell, prepared in Example 2, significantly larger number of live cells (green cells) exists than dead cells (red cells), which indicates excellent cell viability (see FIG. 13 of Experimental Example 10).

### EXAMPLES

Hereinafter, the present invention will be described in more detail.

The following Examples and Experimental Examples are illustrative purpose only, and the scope of the present invention is not limited to the Examples and Experimental Examples.

### Example 1

### Baker's Yeast Coating ([TA-Fe^{III}]₄)

<1-1> Preparation of Experiment

YPAD agar plate: YPAD agar plate was prepared by introducing 20 mL of YPAD agar solution (50 g of YPD broth dissolved in 935 mL of deionized water, 15 g of Bacto™ agar, and 100 mg of adenine hemisulfate) onto a petri dish (area: 87×15 mm).

YPAD broth liquid medium: 50 g of YPD broth and 100 mg of adenine hemisulfate were dissolved in 950 mL of deionized water followed by treatment with autoclave at 121° C. for 15 minutes, and then the resultant was used.

<1-2> Baker's Yeast Coating

A single colony of Saccharomyces cerevisiae, which is a baker's yeast, was taken from the YPAD agar plate, and then cultured in YPAD broth liquid medium at 30° C. for 60 hours with shaking. The yeast cells were washed with deionized water three times, and then dispersed in deionized water. 5 μL of tannic acid (40 mg·mL⁻¹) and 5 μL of FeCl₃.6H₂O (10 mg·mL⁻¹) were sequentially added to the aqueous suspension (490 μL) of the yeast cells with vigorously shaking. After the FeCl₃.6H₂O solution was added, the mixture was vigorously shaked for 10 seconds, and thereafter 0.5 mL of 3-(N-morpholino)propane sulfonic acid (MOPS) buffer (20 mM, pH 7.4) was added to stabilize pH to afford yeast cells coated with stable TA-Fe(III) shells.

Washing was performed three times with deionized water to remove remaining tannic acid and FeCl₃. The coating process, which is started from addition of the tannic acid and FeCl₃.6H₂O through washing with deionized water, was repeated four times.

### Example 2

### HeLa Cell Coating ([TA-Fe^{III}]₄)

<2-1> Preparation Of Experiment

HeLa cells (uterine cervical cancer cell, Korea Cell Line Bank, KCLB No. 10002) were seeded on a cell culture flask together with 10 mL of serum-free Dulbecco's Modified Eagle's medium (DMEM) solution filled with 10% fetal bovine serum (FBS) and 1% penicillin-streptomycin, and then the cells were cultured under 5% CO₂ environment at 37° C.

<2-2> HeLa Cell Coating

When the HeLa cells of Example <2-1> reached confluency of 80% of the area of the cell culture flask, the cells were washed with phosphate buffered saline (PBS) two times. 2 mL of trypsin was added to the cell culture flask, and then the cells were stayed at 37° C. for 5 minutes. When the cells were detached from the flask, 3 mL of DMEM was added. Then, the cells were collected through centrifugation followed by two times of washing with PBS. The detached cells were added to DMEM solution containing 5 μL of tannic acid (0.4 mg·mL⁻¹) and 5 μL of FeCl₃ (0.1 mg·mL⁻¹), and the cells were cultured for 10 seconds to afford HeLa cells which were coated with stable TA-Fe(III) nanoshells.

Washing was performed with deionized water three times to remove remaining tannic acid and FeCl₃. The coating process, which is started from addition of the tannic acid and FeCl₃ through washing with deionized water, was repeated four times.

### Example 3

### Red Blood Cell Coating ([TA-Fe^{III}]₄)

<3-1> Preparation of Red Blood Cells

Red blood cells were prepared from whole blood through centrifugation (3000 rpm, 1500 g, and 10 minutes). To prevent blood clotting, whole blood was centrifuged by using a tube coated with an anticoagulant (such as citrate or heparin), such that blood plasma and buffy coat were placed at supernatant, and red blood cells were placed at pellet. A red blood cell solution was prepared through two times of washing process as follows: plasma and buffy coat were removed; PBS having the same volume as that of red blood cells was added; and the resultant was centrifuged.

<3-2> Red Blood Cell Coating

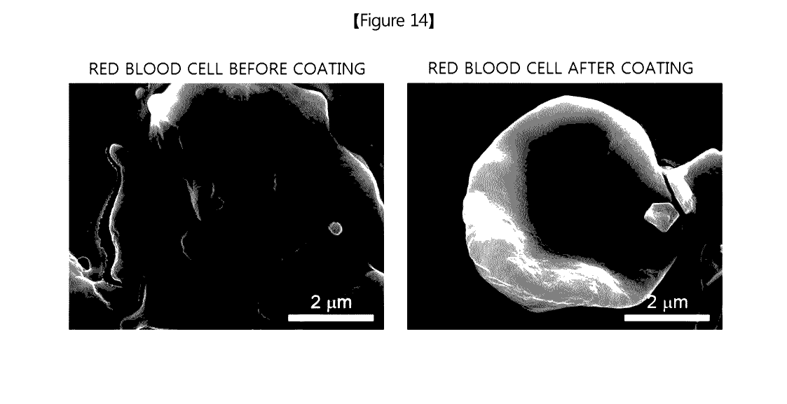

Red blood cells coated with stable TA-Fe(III) nanoshells were obtained by the same method as Experimental Example <2-2> except that red blood cells prepared in Experimental Example <3-1> and PBS were used instead of HeLa cells (uterine cervical cancer cell, Korea Cell Line Bank, KCLB No. 10002); and DMEM solution (see FIG. 14).

### Example 4

### T Lymphocyte Coating ([TA-Fe^{III}]₄)

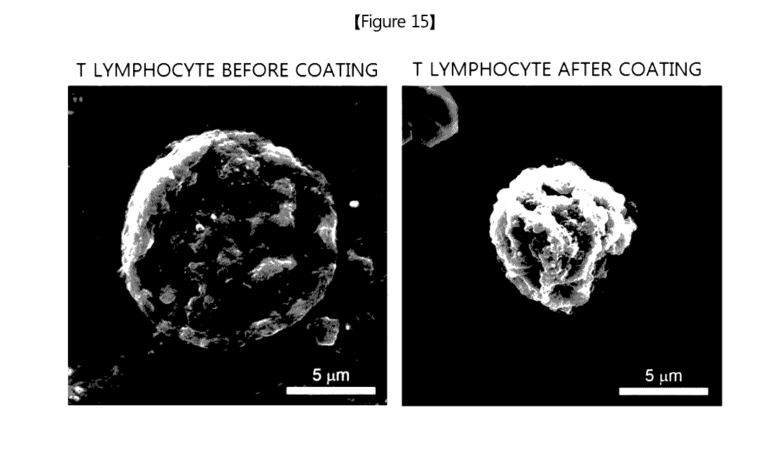

T lymphocytes coated with stable TA-Fe(III) nanoshells were obtained by the same method as Example 2 except that T lymphocytes (Jurkat clone E6-1, Korea Cell Line Bank, KCLB No. 40152) were used instead of HeLa cells (uterine cervical cancer cell, Korea Cell Line Bank, KCLB No. 10002) (see FIG. 15).

### Example 5

### NIH/3T3 Fibroblast Cell Coating ([TA-Fe^{III}]₄)

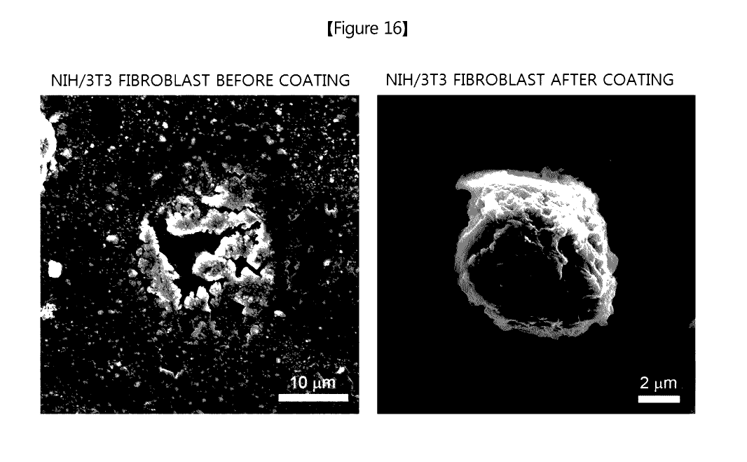

NIH/3T3 fibroblasts coated with stable TA-Fe(III) nanoshells were obtained by the same method as Example 2 except that NIH/3T3 fibroblast cells (Korea Cell Line Bank, KCLB No. 21658) were used instead of HeLa cells (uterine cervical cancer cell, Korea Cell Line Bank, KCLB No. 10002) (see FIG. 16).

### Comparative Example 1

### Baker's Yeast Coating ([TA-Fe^{III}]₂)

Yeast cells coated with unstable TA-Fe(III) shells were obtained by the same method as Experimental Example 1 except that the coating process was performed two times instead of four times.

### Experimental Example 1

### Cell Viability Test 1

To evaluate whether yeast cells prepared in example 1 show excellent viability after coating, the following experiment was conducted, wherein the yeast cells were coated with [TA-Fe^{III}]₄, i.e. TA-Fe(III) shell.

In particular, cell viability was evaluated through fluorescein diacetate (FDA) analysis, wherein the FDA was hydrolyzed by esterase within cells having a metabolic activity, thereby being converted into green fluorescent fluorescein.

Since the FDA was not dissolved in water, an FDA storage solution (5 mg·mL⁻¹) was prepared in acetone. 4 μL of the storage solution for each was mixed with 0.5 mL of baker's yeast cells or yeast cells coated with TA-Fe(III) shells prepared in Example 1. After 20 minutes, the cells were washed three times with deionized water. Thereafter, fluorescent intensities of untreated cells and cell prepared in Example 1 were measured through Synerge™ MX multi-mode microplate reader (BioTekInstruments, USA), and the result was shown in FIG. 2.

FIG. 2 is an image showing a result of evaluating cell viability through Synerge™ MX multi-mode microplate reader (BioTekInstruments, USA) to evaluate whether yeast cells coated with TA-Fe(III) shells prepared in Example 1 show an excellent viability after coating.

As shown in FIG. 2, it has been shown that the yeast cells coated with TA-Fe(III) shells prepared in Example 1 have esterase similar to that of baker's yeast cells which were not treated at all. Particularly, it has been found that resazurin within a cell having a metabolic activity was biologically reduced, to thereby form resorufin so that the fluorescent intensity due to the resorufin in cells of Example 1 was similar to that of baker's yeast cells which were not treated at all.

Thus, the cell coating method according to the present invention may be useful for protecting cells from external environment while keeping the cells alive.

### Experimental Example 2

### Evaluation of Presence and Absence of Cell Coating 1

To evaluate whether [TA-Fe^{III}]₄, i.e. TA-Fe(III) shell prepared in Example 1, is well coated on yeast cells, a raman spectrum was obtained by using Jobin Yvon/HORIBA LabRAM spectrometer equipped with a microscopy (Olympus BX 41, Japan), and the result was shown in FIG. 3.

FIG. 3 is a raman spectrum image to evaluate whether the TA-Fe(III) shell prepared in Example 1 is well coated on yeast cells.

As shown in FIG. 3, it has been found that strong bands appeared at 1354 cm⁻' and 1482 cm⁻' wavelength areas, indicating TA having a ring structure coated on the yeast cells, and therefore the TA-Fe(III) shell prepared in Example 1 was well coated on the yeast cells.

### Experimental Example 3

### Evaluation Of Presence And Absence Of Cell Coating 2

To evaluate whether [TA-Fe^{III}]₄, which was TA-Fe(III) shell prepared in Example 1, is well coated on yeast cells, a structure was observed through scanning electron microscopy (SEM) and transmission electron microscopy (TEM). In particular, the SEM image was observed by using FEI Inspect F50 microscopy (FEI, Netherlands) (acceleration voltage: 10 kV), and the TEM image was observed by using JEM-2100 (JEOL, Japan). The result was shown in FIG. 4.

FIG. 4 is an image showing a structure observed through SEM and TEM images to evaluate whether the TA-Fe(III) shell prepared in Example 1 is well coated on yeast cells.

As shown in the SEM image of FIG. 4, it has been shown that, comparing to untreated cells, homogeneous TA-Fe(III) shells were formed on overall cells of Example 1. Additionally, as shown in the TEM image of FIG. 4, by dissecting the cells to observe a cross section, it has been found that the average thickness of the TA-Fe(III) shells was 40 nm.

### Experimental Experiment 4

### Evaluation of Cell Aggregation due to E. coli

To evaluate whether, after coating, cell aggregation due to E. coli occurs in yeast cells coated with [TA-Fe^{III}]₄, i.e. TA-Fe(III) shell, prepared in Example 1, the following experiment was conducted.

Prior to performing the experiment, it has been found that zeta potential of untreated cells was −9 mV and zeta potential of the yeast cells coated with TA-Fe(III) shells prepared in Example was −18 mV through microelectrophoresis measurement using Zetasizer Nano ZS (Malvern, UK) based on Smoluchowski model. Thus, it has been expected that, comparing to untreated cells, unnecessary self-aggregation of cells of Example 1 is efficiently prevented due to increased repulsive power between negative charges.

To express fimbriae of E. coli, a single colony of MG1655, which is a wild type strain of E. coli, was inoculated into Luria-Bertani broth (LB, Difco) liquid medium followed by culturing at 37° C. for 16 hours. The E. coli was centrifuged (5000 g, 5 minutes, 4° C.), washed with deionized water two times, and then dispersed in PBS (pH 7.4). Optical densities at 600 nm for cells and E. coli were, respectively, 5 and 3 (wherein, the optical density was measured by using Ultrospec 7000 spectrophotometer (GE Healthcare Life Science, UK)). The cells and E. coli suspensions (20 μL for each) were mixed together. After one minute, presence and absence of cell aggregation was observed through LSM 700 confocal laser-scanning microscopy (Carl Zeiss, Germany). The result was shown in FIG. 5.

FIG. 5 is an image showing presence and absence of cell aggregation observed through LSM 700 confocal laser scanning microscopy (Carl Zeiss, Germany) to evaluate whether cell aggregation due to E. coli occurs in yeast cells coated with TA-Fe(III) shell prepared in Example 1 after coating.

As shown in FIG. 5, it has been shown that, for untreated cells, cell aggregation due to E. coli occurred, whereas, for the cells prepared in Example 1, cell aggregation did not occur due to increased surface negative charges.

### Experimental Example 5

### Protein Conjugation Ability Test

To evaluate protein conjugation ability of [TA-Fe^{III}]₄, i.e. TA-Fe(III) shell prepared in Example 1, the following experiment was conducted.

BSA-Alexa 647 (0.4 mg·mL⁻¹, Life Technologies), which was a protein conjugated with a chromophore, was added to and mixed with aqueous suspension of untreated cells or cells prepared in Example 1, and the resultant was cultured for 15 minutes. Thereafter, protein conjugation ability was measured through LSM 700 confocal laser-scanning microscopy (Carl Zeiss, Germany). The result was shown in FIG. 6.

FIG. 6 is a confocal laser-scanning microscopy (Carl Zeiss, Germany) image observed after inducing binding of cells to BSA-Alexa 647 (0.4 mg·mL⁻¹, Life Technologies), which is a protein conjugated with a chromophore, to evaluate a protein conjugation ability of the TA-Fe(III) shell prepared in Example 1.

As shown in FIG. 6, for untreated cells, there was no shell capable of binding to BSA-Alexa 647, which is a protein conjugated with a chromophore, so that fluorescence was not observed, whereas, for the cells prepared in Example 1, TA-Fe(III) shells bound to BSA-Alexa 647, so that fluorescence was observed. Thus, excellent protein conjugation ability was demonstrated.

Then, in addition to the protein conjugation ability of the cells prepared in Example 1, to evaluate viability, FDA (Sigma) analysis was performed. Since the FDA was not dissolved in water, an FDA storage solution (5 mg·mL⁻¹) was prepared in acetone. 4 μL of the storage solution for each was mixed with 0.5 mL of baker's yeast cells or the yeast cells coated with TA-Fe(III) shells prepared in Example 1. After 20 minutes, the cells were washed three times with deionized water. The cells were observed through LSM 700 confocal laser-scanning microscopy (Carl Zeiss, Germany), and the result was shown in FIG. 7.

FIG. 7 is an image observed through confocal laser-scanning microscopy (Carl Zeiss, Germany) after inducing binding of cells to BSA-Alexa 647 (0.4 mg·mL⁻¹, Life Technologies), which is a protein conjugated with a chromophore, and adding FDA (Sigma) to evaluate a protein conjugation ability of TA-Fe(III) shell prepared in Example 1, and also evaluate viability of the cells coated with the shell.

As shown in FIG. 7, it has been found that a core-shell structure for live cells was shown, so that the cells prepared in Example 1 had excellent viability, as well as protein conjugation ability.

### Experimental Example 6

### Evaluation of Ability to Regulate Cell Differentiation

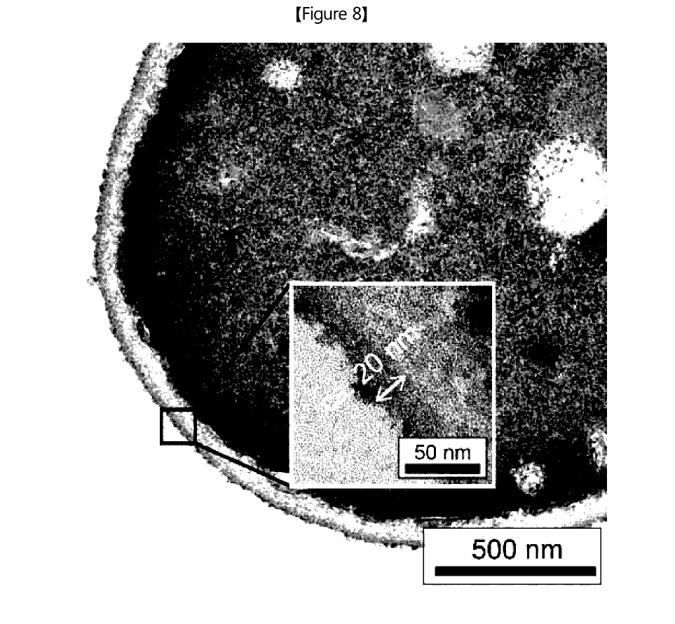

To evaluate whether cell differentiation ability of yeast cells prepared in example 1 is regulated after coating, following experiments were conducted, wherein the yeast cells were coated with [TA-Fe^{III}]₄, i.e. TA-Fe(III) shell. For Comparative Example, [TA-Fe^{III}]₂, i.e. TA-Fe(III) shell prepared in Comparative Example 1, was used (it has been found that the shell thickness was about 20 nm through the TEM image which was shown in FIG. 8).

FIG. 8 is a cross section image of [TA-Fe^{III}]₂, i.e. TA-Fe(III) shell prepared in Comparative Example 1 observed through a TEM image and indicating a thickness of about 20 nm.

Prior to performing the experiment, optical densities at 600 nm of untreated cells, and cells prepared in Comparative Example or Example 1 were adjusted to 2.0 by using deionized water (Ultrospec 7000 spectrophotometer (GE Healthcare Life Science, UK)). To degrade the TA-Fe(III) shell, the yeast cells (OD₆₀₀=2.0) were treated with 5 or 20 mM of HCl for 90 minutes.

(a) agar plate: The yeast cells were washed with deionized water three times, and then dispersed in deionized water. A colony-forming unit (CFU) value was obtained by culturing the cells in the YPAD agar plate. 15 μL of yeast suspension was diluted in deionized water such that the suspension became 300 μL, and then 10 μL of the diluted suspension was further diluted such that the suspension became 1 mL (total dilution factor: 2000). 150 μL of the final yeast suspension was spread on YPAD agar plate. The plate was cultured in 2-D in a heat incubator, and then colonies were listed. Thereafter, the CFU value was converted into a log value.

(b) Liquid medium: A value was calculated based on a linear fitted graph of lnOD₆₀₀ vs. time. Untreated cells and the cells prepared in Example 1 (OD₆₀₀=0.01), respectively, were suspended in YPAD broth liquid medium followed by culturing in a mixing incubator at 30° C. 400 μL of the mixture was taken at precalculated time, and then optical density was measured at 600 nm through UV-visible spectroscopy. A growth curve for HCl-treated cells, which were prepared in Example 1, was obtained by the same protocol.

The result was shown in FIG. 9.

FIG. 9 is an image showing a result of conducting an experiment to evaluate whether cell differentiation ability of yeast cells prepared in example 1 is regulated after coating, wherein the yeast cells were coated with [TA-Fe^{III}]₄, i.e. TA-Fe(III) shell.

As shown in the solid phase agar plate of FIG. 9, it has been shown that, before an acid was added, the CFU value of cells coated with the shell [TA-Fe^{III}]₄ (thickness of about 40 nm) prepared in Example 1 was significantly lower than those of untreated cells and Comparative Example 1 cells [TA-Fe^{III}]₂ (thickness of about 20 nm), indicating that the cell differentiation ability was readily inhibited. In addition, it has been found that, when the shell prepared in Example 1 was degraded according to addition of an acid, cell differentiation the same as that of untreated cells occurred.

Additionally, as shown in liquid-phase culture (liquid medium) of FIG. 9, it has been shown that time required to achieve InOD₆₀₀ of −2 increased in proportion to the thickness of the shell coated on cells. Namely, it has been found that, before the shell was degraded, cell differentiation of the cells coated with the shell [TA-Fe^{III}]₄ (thickness of about 40 nm) prepared in Example 1 was significantly inhibited comparing to those of untreated cells and Comparative Example 1 cells.

Thus, the composition for cell coating according to the present invention protects cell from external environment while keeping the cells alive, and also the composition is readily degraded as necessary while protecting cells.

### Experimental Example 7

### Evaluation of Cell Protecting Ability from UV Irradiation

To evaluate whether yeast cells prepared in Example 1 were entirely protected from UV irradiation after coating, the following experiment was conducted, wherein the yeast cells were coated with [TA-Fe^{III}]₄, i.e. TA-Fe(III) shell.

Firstly, optical density of untreated baker's yeast cells or the cells prepared in Example 1 was adjusted to 1.0, and thereafter 3 mL of the cell suspension was prepared in a quartz cuvette (Hellma Co., Germany). The cuvette was placed in a chamber type shield box equipped with 4-W filter UV lamp VL-4.LC (Vilber Lourmat Co., France). A distance between the cuvette and UV lamp was adjusted to 5 cm. UV-C light (λ: 254 nm) was irradiated for predetermined time (irradiated energy: 8 J or 12 J). The irradiated energy was calculated based on the UV lamp intensity previously known (intensity at 15 cm=265 μW·cm⁻²).

After UV-C was irradiated, FDA analysis was performed by the same method as <Experimental Example 5> to evaluate final yeast cell viability (the analysis was performed on at least 300 cells). The result was shown in FIG. 10.

FIG. 10 is an image showing a result of evaluating whether cells are entirely protected from UV irradiation after the yeast cells are coated with [TA-Fe^{III}]₄, i.e. TA-Fe(III) shell, prepared in Example 1.

As shown in FIG. 10, comparing to untreated cells, it has been shown that cell viability from UV irradiation of the cells prepared in Example 1 was significantly increased due to [TA-Fe^{III}]₄, i.e. TA-Fe(III) shell. In particular, when light having the intensity of 12 J was irradiated, it has been shown that viability of untreated cells was about 9%, whereas viability of cells (i.e. cells prepared in Example 1) coated with [TA-Fe^{III}]₄, i.e. TA-Fe(III) shell, was 70% or more.

### Experimental Example 8

### Evaluation of Cell Protecting Ability from Silver Nanoparticles

To evaluate whether yeast cells prepared in Example 1 were entirely protected from silver nanoparticles after coating, the following experiment was conducted, wherein the yeast cells were coated with [TA-Fe^{III}]₄, i.e. TA-Fe(III) shell.

Firstly, optical density at 600 nm of untreated baker's yeast cells or the cells prepared in Example 1 was adjusted to 2.0, and the cells were diluted with deionized water. The yeast suspensions (500 μL per each) were respectively mixed with 500 μL of silver nanoparticle suspension (0.02 mg·mg⁻¹) having three different diameters (which were 20 nm, 60 nm, or 100 nm). The mixture was concentrated to 50 μL through centrifugation (10,000 rpm, 1 minute), and the concentrate was cultured at room temperature for 20 hours with careful shaking. The yeast cells were stained with FDA (4 μL·mL⁻¹ of storage solution corresponding to 5 mg·mL⁻¹ dissolved in acetone) and propidium iodide (PI) (2 μL·mL⁻¹ of storage solution corresponding to 1 mg·mL⁻¹ dissolved in deionized water), and the cells were observed through LSM 700 confocal laser-scanning microscopy (Carl Zeiss, Germany), wherein the FDA stained live cell and the PI stained dead cells or dying cells. The result was shown in FIG. 11.

FIG. 11 is an image showing a result of evaluating whether cells are entirely protected from silver nanoparticles after the yeast cells are coated with [TA-Fe^{III}]₄, i.e. TA-Fe(III) shell prepared in Example 1.

As shown in FIG. 11, comparing to untreated cells, it has been shown that cell viability from silver nanoparticles of the cells prepared in Example 1 was significantly increased due to [TA-Fe^{III}]₄, i.e. TA-Fe(III) shell. In particular, it has been found that, when silver nanoparticles (diameter: 20 nm, 60 nm, or 100 nm) were added, untreated cells showed cell death (%) of about 28% or more, whereas cells (i.e. cells prepared in Example 1) coated with [TA-Fe^{III}]₄, i.e. TA-Fe(III) shell, showed cell death (%) of about 11% or less.

### Experimental Example 9

### Evaluation of Presence and Absence of Cell Coating 3

A structure was observed through a SEM image to evaluate whether quality of coating in HeLa cells coated with [TA-Fe^{III}]₄, i.e. TA-Fe(III) shell prepared in Example 2. In particular, the SEM was image was obtained through FEI Inspect F50 microscopy (FEI, Netherlands) (accelerating voltage: 10 kV). The result was shown in FIG. 12.

FIG. 12 is an image showing a structure observed through the SEM image to evaluate whether quality of coating in HeLa cells coated with [TA-Fe^{III}]₄, i.e. TA-Fe(III) shell prepared in Example 2.

As shown in the SEM image, it has been shown that, comparing to untreated cells, homogeneous TA-Fe(III) shells were formed on the overall HeLa cells of Example 2.

### Experimental Example 10

### Cell Viability Test 2

It has been evaluated whether the HeLa cells prepared in Example 2 show excellent viability after coating through Live/Dead® viability/cytotoxicity kit (Life Technologies), wherein the HeLa cells were coated with [TA-Fe^{III}]₄, i.e. TA-Fe(III) shell. The result was shown in FIG. 13.

FIG. 13 is an image of evaluating whether HeLa cells prepared in Example 2 show excellent viability after coating through Live/Dead® viability/cytotoxicity kit (Life Technologies), wherein the HeLa cells were coated with [TA-Fe^{III}]₄, i.e. TA-Fe(III) shell.

As shown in FIG. 13, it has been shown that, for the HeLa cells coated with [TA-Fe^{III}]₄, i.e. TA-Fe(III) shell, prepared in Example 2, remarkably larger numbers of live cells (green cells) existed than dead cells (red cells), indicating excellent cell viability.

What is claimed is:

1. A cell coating method comprising: (step 1) adding, to an aqueous solution including cells, a compound including one or more gallate groups expressed by Chemical Formula 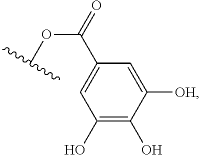 [SMILES: *OC(=O)c1cc(O)c(O)c(O)c1]
and a lanthanoid metal salt or transition metal salt.

2. The cell coating method of claim 1, wherein the cells of step 1 are yeast cells, mammalian cells or immune cells having a metabolic activity.

3. The cell coating method of claim 1, wherein the cells of step 1 are Saccharomyces cerevisiae cells, HeLa cells, red blood cells, T lymphocytes, NIH/3T3 fibroblast cells, Escherichia coli cells, Bacillus subtilis cells, Lactobacillus spp. cells, Streptococcus spp. cells, Bifidobacterium cells, Cyanobacteria cells, Spirulina cells, Chlorella cells, mesenchymal stem cells, osteoblasts, chondrocytes, B lymphocytes, Langerhans cells, neuron cells, keratinocytes, hepatocytes, human vascular endothelial cells, Chinese hamster ovary cells, islets of Langerhans or endocrine cells.

4. The cell coating method of claim 1, wherein the compound comprising the gallate group is at least one selected from the group consisting of tannic acid (TA), gallic acid, theaflavin-3-gallate, epigallocatechin gallate and epicatechin gallate.

5. the cell coating method of claim 1, wherein the metal of the lanthanoid metal salt is at least one selected from the group consisting of cerium (Ce), europium (Eu), gadolinium (Gd) and terbium (Tb); and
the metal of the transition metal salt is at least one selected from the group consisting of aluminum (Al), vanadium (V), manganese (Mn), ferrous (Fe), zinc (Zn), zirconium (Zr), molybdenum (Mo), ruthenium (Ru) and rhodium (Rh).

6. The cell coating method of claim 1, further comprising: (step 2) purifying coated cells through centrifugation after step 1.

7. The cell coating method of claim 6, wherein the cell coating method is repetitively performed two to six times to adjust a thickness of the coating.

8. A cell having a surface coated with a nanoshell comprising a compound including one or more gallate groups expressed by Chemical Formula 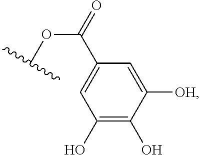 [SMILES: *OC(=O)c1cc(O)c(O)c(O)c1]
and lanthanoid metal ion or transition metal ion.

9. The cell of claim 8, wherein the lanthanoid metal ion or transition metal ion is capable of forming a complex with the gallate group expressed by Chemical Formula

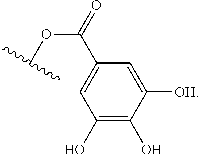 [SMILES: *OC(=O)c1cc(O)c(O)c(O)c1]

10. The cell of claim 8, wherein a thickness of the nanoshell is between 25 to 55 nm.

11. A cell protecting method comprising: coating the cell using the method of claim 1.

12. The cell protecting method of claim 11, wherein the cell protecting method is characterized in that cells are kept alive and protected from external environment by a film coated on the cell surface.

13. A cell proliferation inhibiting method comprising:
coating the cell using the method of claim 1.

14. The cell proliferation inhibiting method of claim 13, wherein the cell proliferation inhibiting method is characterized in that cells are kept alive and protected from external environment by a film coated on the cell surface.
